(19) United States
(12) Patent Application Publication
(10) Pub. No.: US 2025/0014384 A1
(43) Pub. Date: Jan. 9, 2025

(54) METHOD AND DEVICE FOR AUTHENTICATING A DRIVER OF A VEHICLE

(71) Applicant: GRABTAXI HOLDINGS PTE. LTD., Singapore (SG)

(72) Inventors: Munirul ABEDIN, Sammamish, WA (US); Mohd Nazri Bin RAMLIY, Petaling Jaya (MY); Kai Lin LAI, Singapore (SG); Ren Xian THIAN, Petaling Jaya (MY); Naveen AGGARWAL, Singapore (SG)

(21) Appl. No.: 18/711,225

(22) PCT Filed: Nov. 21, 2022
(86) PCT No.: PCT/SG2022/050847

§ 371 Date: May 17, 2024

(30) Foreign Application Priority Data
Dec. 1, 2021 (SG) 10202113347S

(51) Int. Cl. G06V 40/16; G06Q 50/40; G06V 20/50

(52) U.S. Cl. CPC G06V 40/16; G06Q 50/40; G06V 20/50

(57) ABSTRACT

Aspects concern a method for authenticating a driver of a vehicle, comprising sending a request to a mobile phone to provide a selfie of a user of the mobile phone by means of the mobile phone, wherein the mobile phone is registered to receive orders for transport tasks, determining a time the user takes to provide a selfie in response to the request, sending additional requests to the mobile phone to provide selfies by means of the mobile phone if the determined time exceeds a predetermined threshold with a higher frequency than if the determined time does not exceed the predetermined threshold and performing authentication of the user as legitimate driver using selfies provided in response to the additional requests.

### TECHNICAL FIELD

Various aspects of this disclosure relate to methods and devices for authenticating a driver of a vehicle.

### BACKGROUND

Whether customers are satisfied with an e-hailing service which enables customers to hail taxis using their smartphones largely depends on the quality of the e-hailing service's drivers, i.e. whether they take sensible routes, do not try to cheat the customers and are friendly. To have control over the quality of the drivers, an e-hailing server may maintain a database storing information of a driver, such as whether the driver is white listed or blacklisted for the e-hailing service. This is done by giving each driver an account and storing information in association with the account. However, it may occur that a driver lets the driver's account be used by another driver, i.e. multiple persons may share the same account.

Driver account sharing is a common problem in e-hailing and similar transport services (which may also be for food, parcels etc.). Driver account sharing here means that a driver has an account with the transport service's operator to sign up (register) for taking transport tasks but another person drives, i.e. performs the transport tasks. The actual driver is thus not properly authenticated (because the driver uses the account of another driver). This creates security and safety issues. There are a wide variety of reasons why this happens, e.g. the same vehicle is being driven by multiple people, or additional drivers do not intend to go through rigorous background checks, or simply the original driver (i.e. for whom the account was created) is outsourcing the job for monetary gains. Regardless of intent, an alternate driver is a safety risk for the transport service's operator and it erodes passenger trust in that the transport service's operator ensures drivers are properly vetted.

Therefore, approaches to reliably authenticate drivers of vehicles of a transport service are desirable.

### SUMMARY

Various embodiments concern a method for authenticating a driver of a vehicle, including sending a request to a mobile phone to provide a selfie of a user of the mobile phone by means of the mobile phone, wherein the mobile phone is registered to receive orders for transport tasks, determining a time the user takes to provide a selfie in response to the request, sending additional requests to the mobile phone to provide selfies by means of the mobile phone if the determined time exceeds a predetermined threshold with a higher frequency than if the determined time does not exceed the predetermined threshold and performing authentication of the user as legitimate driver using selfies provided in response to the additional requests.

According to one embodiment, the method includes, if the determined time exceeds the predetermined threshold, triggering the sending of an additional request at each of a set of predetermined events.

According to one embodiment, the method includes avoiding triggering sending an additional request by the predetermined events if the determined time does not exceed the predetermined threshold.

According to one embodiment, the predetermined events includes the user toggling an availability state for taking transport tasks from not available to available and/or the user having completed a transport task.

According to one embodiment, the predetermined threshold is in the range of 5 minutes to 30 minutes (e.g. 15 minutes).

According to one embodiment, the mobile phone is registered under a user account to receive orders for transport tasks and the method includes flagging the user account as suspicious if the determined time exceeds the predetermined threshold and sending additional requests to the mobile phone if the account is flagged as suspicious than if the account is not flagged as suspicious.

According to one embodiment, the method includes flagging the account as suspicious if a number of passengers which report a different vehicle or a different driver for their rides than expected according to the user's profile within a first predetermined period exceeds a first further predetermined threshold and/or including flagging the account as suspicious if the frequency with which different mobile phones have been used to register under the user account within a second predetermined period exceeds a second further predetermined threshold.

According to one embodiment, the method includes sending the additional requests to the mobile phone with a minimum time buffer period between the sending of the additional requests.

According to one embodiment, the time buffer period is in the range of 5 minutes to 30 minutes.

According to one embodiment, the method includes sending an additional request after a predetermined request interval if the determined time does not exceed the predetermined threshold.

According to one embodiment, an authentication server is provided configured to perform the method of one of the embodiments described above.

According to one embodiment, a computer program element is provided including program instructions, which, when executed by one or more processors, cause the one or more processors to perform the method of one of the embodiments described above.

According to one embodiment, a computer-readable medium is provided including program instructions, which, when executed by one or more processors, cause the one or more processors to perform the method of one of the embodiments described above.

### BRIEF DESCRIPTION OF THE DRAWINGS

The invention will be better understood with reference to the detailed description when considered in conjunction with the non-limiting examples and the accompanying drawings, in which:

FIG. 1 shows a communication arrangement for usage of an e-hailing service including a smartphone and a server.

FIG. 2 shows an arrangement for taking transport tasks.

FIG. 3 illustrates results of a data analysis (for an e-hailing service) with regard to patterns how long drivers take to respond to a selfie request.

FIG. 4 shows a histogram of gap length between two selfie events.

FIG. 5 shows two examples of data sequences related to selfie authentication.

FIG. 6 illustrates a process to request selfies depending on a driver being suspicious due to having taken too long to react to an earlier selfie request.

FIG. 7 shows a flow diagram illustrating a method for authenticating a driver according to an embodiment.

FIG. 8 shows an authentication server according to an embodiment.

### DETAILED DESCRIPTION

The following detailed description refers to the accompanying drawings that show, by way of illustration, specific details and embodiments in which the disclosure may be practiced. These embodiments are described in sufficient detail to enable those skilled in the art to practice the disclosure. Other embodiments may be utilized and structural, and logical changes may be made without departing from the scope of the disclosure. The various embodiments are not necessarily mutually exclusive, as some embodiments can be combined with one or more other embodiments to form new embodiments.

Embodiments described in the context of one of the devices or methods are analogously valid for the other devices or methods. Similarly, embodiments described in the context of a device are analogously valid for a vehicle or a method, and vice-versa.

Features that are described in the context of an embodiment may correspondingly be applicable to the same or similar features in the other embodiments. Features that are described in the context of an embodiment may correspondingly be applicable to the other embodiments, even if not explicitly described in these other embodiments. Furthermore, additions and/or combinations and/or alternatives as described for a feature in the context of an embodiment may correspondingly be applicable to the same or similar feature in the other embodiments.

In the context of various embodiments, the articles “a”, “an” and “the” as used with regard to a feature or element include a reference to one or more of the features or elements.

As used herein, the term “and/or” includes any and all combinations of one or more of the associated listed items.

In the following, embodiments will be described in detail.

An e-hailing app, typically used on a smartphone, allows its user to hail a taxi (or also a private driver) through his or her smartphone for a trip.

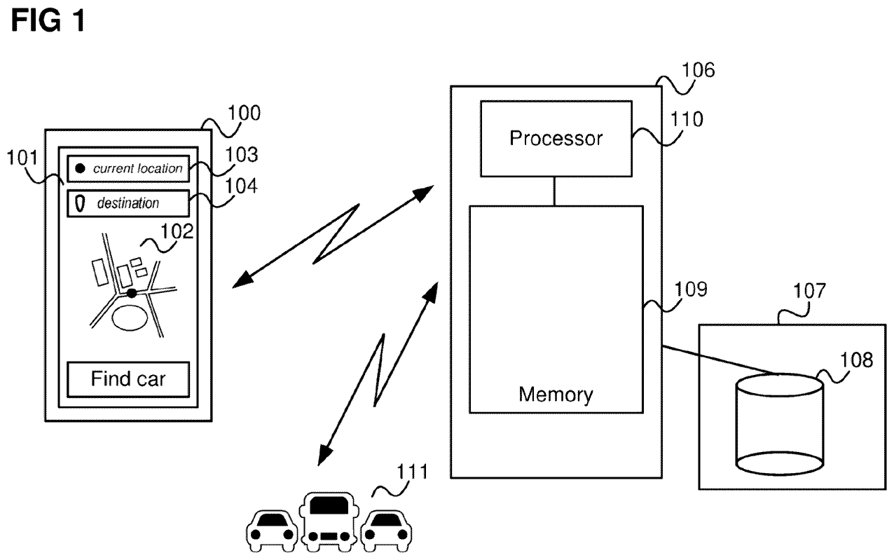

FIG. 1 shows a communication arrangement including a smartphone 100 and a server (computer) 106.

The smartphone 100 has a screen showing the graphical user interface (GUI) of an e-hailing app that the smartphone's user has previously installed on his smartphone and has opened (i.e. started) to e-hail a ride (taxi or private driver).

The GUI 101 includes a map 102 of the vicinity of the user's position (which the app may determine based on a location service, e.g. a GPS-based location service). Further, the GUI 101 includes a box for point of departure 103 (which may be set to the user's present location obtained from location service) and a box for destination 104 which the user may touch to enter a destination (e.g. opening a list of possible destinations). There may also be a menu (not shown) allowing the user to select various options, e.g. how to pay (cash, credit card, credit balance of the e-hailing service). When the user has selected a destination and made any necessary option selections, he or she may touch a “find car” button 105 to initiate searching of a suitable car.

For this, the e-hailing app communicates with the server 106 of the e-hailing service via a radio connection. The server 106 may consult a memory 109 or a data storage 108 having information about the current location of registered vehicles 111, about when they are expected to be free, about traffic jams etc. From this, a processor 110 of the server 106 selects the most suitable vehicle (if available, i.e. if the request can be fulfilled) and provides an estimate of the time when the driver will be there to pick up the user, a price of the ride and how long it will take to get to the destination. The server communicates this back to the smartphone 100 and the smartphone 100 displays this information on the GUI 101. The user may then accept (i.e. book) by touching a corresponding button. If the user accepts, the server 106 informs the selected vehicle 111 (or, equivalently, its driver), i.e. the vehicle the server 106 has allocated for fulfilling the transport request.

It should be noted while the server 106 is described as a single server, its functionality, e.g. for providing an e-hailing service for a whole city including distribution of transport tasks, driver authentication etc., will in practical application typically be provided by an arrangement of multiple server computers (e.g. implementing a cloud service). Accordingly, the functionality described in the following provided by the server 106 may be understood to be provided by an arrangement of servers or server computers.

The data storage 108 may for example be part of a cloud-based system 107 provided by a cloud storage provider to store and access data which it may use for taking decisions, such as information about the location of passengers and vehicles, their history (earlier bookings and routes taken) etc.

The server 106 together with the vehicles 111 provides the e-hailing service, i.e. forms a transport system.

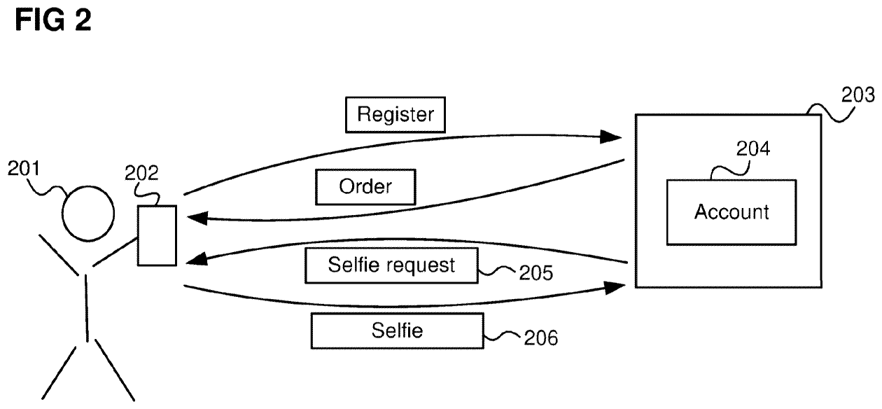

To receive orders for transport tasks, the driver of a vehicle uses a mobile device, usually a mobile phone (smartphone), as illustrated in FIG. 2.

FIG. 2 shows an arrangement for taking transport tasks.

A driver 201 of a vehicle 111 has a mobile phone 202 by which the driver 201 signs up (registers) at a server 203 (e.g. corresponding to server 106). The driver 201 signs up under an account 204 which the driver 201 has made earlier 204 with the server 203 (by subscription). By signing up, the driver 201 indicates that he or she wants to be provided with orders for transport tasks. In response, the server 203 provides transport tasks to the mobile phone 202 that the driver 201 may accept and perform using his or her vehicle. The server 203 and the mobile phone 202 may communicate using a mobile communication network.

To ensure safety and security of the e-hailing service (generally the transport system) drivers should be properly authenticated. As mentioned, the server 203 stores (e.g. in data base 108), an account 204 for each driver. A driver 201 signs up with the server 203 to drive a certain vehicle 111. However, accounts may be shared, i.e. an account of a certain driver may be used to sign up for driving a vehicle 111 but another (i.e. alternate or fake) driver actually drives the vehicle. Since, for example, the alternate driver may be a much worse driver or even a criminal, this creates safety and security issues for the transport system.

Detecting whether a vehicle is being driven by an alternate driver is not easy when high effort (like constantly monitoring a vehicle's interior with video) should be avoided. In particular, passenger reports of driver faces not matching their profile pictures are voluntary and typically sparse and are only received after the transport task has been performed.

In the following, approaches are provided for detecting possible alternate drivers and mechanisms to curb account sharing are given.

The approaches described in the following are based on selfie verification. This means that for authenticating the driver 201, the server 203 requests the driver (by a selfie request 205 sent to the driver's mobile phone 202) to provide a photo of him or herself (i.e. a self-portrait) by means of the mobile phone 202 via which the driver also receives orders for transport tasks. In other words, the driver 201 is requested to provide a selfie 206 to the server 203. The server 203 (e.g. operating as transport system controller) may then compare the selfie with a reference picture associated with (e.g. included in) the account 204 that is being used by the driver such that it can be verified that the account 204 to which transport task orders are sent is used by the driver to which the account belongs. This is for example being performed by an authentication server part or function of the server 203.

In a typical e-hailing service, drivers are repeatedly subjected to selfie verification e.g. at least once in every 8-72 hours. While selfie verification allows verifying that the face of the person currently holding the device (to which orders are sent) matches the reference picture of the account used by the device, an account's owner can hand over the device to another person after taking the selfie (i.e. after responding to the selfie request). A driver knows that it will be another 8-72 hours before he or she is asked for a selfie again.

Selfie verification (or validation) may for example be implemented as the following process: a backend service logic (e.g. of server 106) determines whether a driver 201 should be asked for a selfie. If the result is “yes”, the server 203 sends a request 205 to the driver's device (i.e. smartphone) 202 and the driver is presented with a popup that he or she needs to take a selfie since otherwise, the driver 201 will not be able to take a next job (i.e. transport task). The server 203 compares received selfies with the reference picture in the corresponding account 204 and authenticates the driver's device 202 if the selfies match the reference pictures. If a selfie does not match the reference picture in the account 204 for which the selfie was requested, the selfie verification has failed and transport tasks can no longer be accepted with (or are no longer provided to) the device 202 using the account 204. Accordingly, the selfie verification process has the following four 4 states: Selfie_Requested, Selfie_Verified, Selfie_Failed, Selfie_UnknownState (a rarely occurring error state).

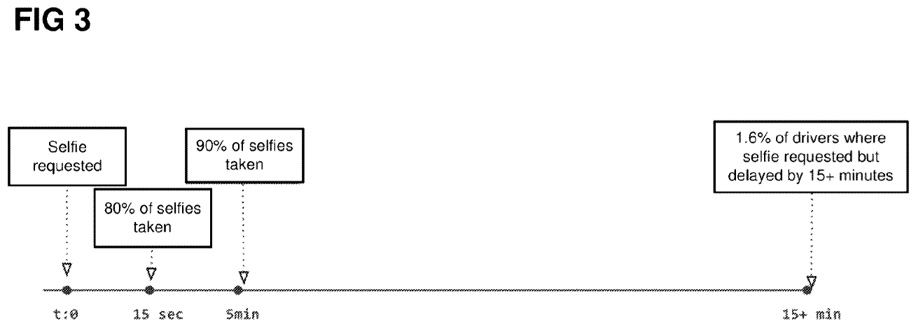

FIG. 3 illustrates results of a data analysis (for an e-hailing service) with regard to patterns how long drivers take to respond to a selfie request.

In 90% of cases (i.e. selfie requests) the respective driver provides the selfie within 5 minutes. However, there is a long tail in the distribution, particularly 1.6% of cases where the respective driver saw the selfie request and decided not to do it at that time. Instead it took them 15 minutes to 12 hours or more before they could perform the validation. Exemplary data shows that for certain drivers this is a routine occurrence.

Various embodiments are based on the assumption that these extreme cases (i.e. the 1.6% indicated in FIG. 3) are cases where a driver (driver #2) has the smartphone but is a different individual than another driver (driver #1) whose reference picture is in the account for that phone (i.e. the legitimate driver). The driver #2 knows that if he or she proceeds with taking a selfie in response to the selfie request authentication would fail. Therefore, driver #2, when having received the selfie request, drives back to driver #1, acquires the correct selfie from driver #1 and continues accepting transport tasks with an authenticated device for 8 hours or longer.

For data analysis, each execution of the selfie verification process is represented by a “selfie sequence” of the above states.

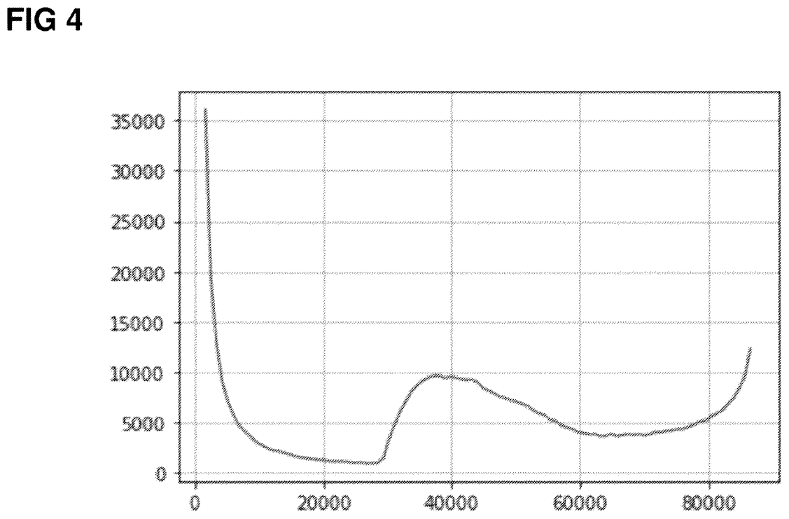

To analyze data about selfie verification based on selfie sequences it is necessary to identify beginnings and ends of the sequences. It is reasonable that a selfie sequence starts with Selfie_Requested state, however termination or delimiters are not as apparent. Studying exemplary e-hailing self verification data shows, as illustrated in FIG. 3, that the gap between two consecutive selfie events (i.e. changes of state) is usually small, i.e. there is a quick drop in a gap histogram, until it picks up again after 8 hours (28800 sec in FIG. 4).

FIG. 4 shows a histogram of gap length between two selfie events.

The pick-up is in this example due to selfie verification being repeated after 8 hours. Thus, in such as case (8 hours selfie verification interval) is a good terminating condition for selfie sequences (i.e. selfie sequences are assumed to be 8 hours or shorter).

Further, for data analysis based on selfie sequences, sequences are for example bucketed into Fibonacci time segments (observing the power law nature of the curve of FIG. 4). Otherwise sequence processing becomes too complex and expensive for the analysis task at hand. Once bucketed, two strategies may be applied: last selfie state as the bucket's overall state, or establishing precedence and setting the precedent state as the bucket's state. There is not a huge difference between the two and in the following, the latter is used. Fibonacci bucket ranges are as follows (given in minutes) [0.25, 0.5, 0.75, 1.25, 2.0, 3.25, 5.25, 8.5, 13.75, 22.25, 36.0, 58.25, 94.25, 152.5, 246.75, 399.25] i.e. 16 slots starting with 15 sec, 30 sec, 45 sec, 1 m 15 sec, . . . .

In an exemplary data set, 2,266,342 such sequences were observed.

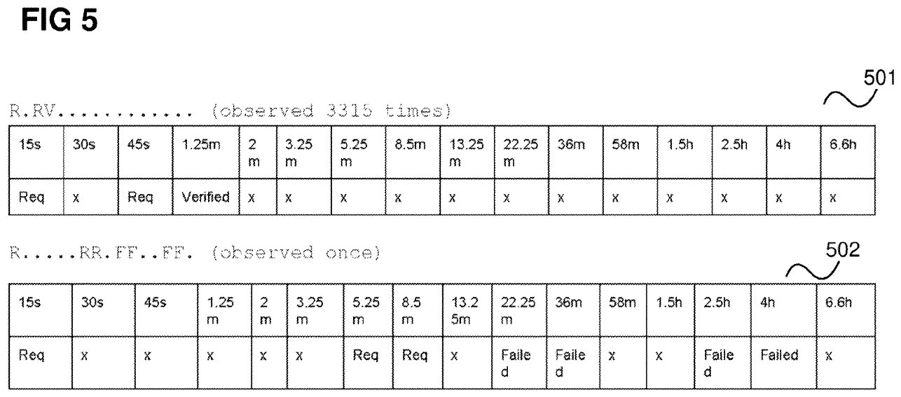

FIG. 5 shows two examples 501, 502 of data sequences according to the above segmentation into Fibonacci time segments.

In the exemplary data, 80% of all selfie sequences are of the type ‘V...............’ where the driver provides a selfie in under 15 seconds. Further, 90% are within 5 minutes. Switching from sequences to drivers, it can be seen that 93% of the drivers regularly provide a selfie within a few minutes of the request while 1.6% of the drivers almost always seem to require a long gap, 15 minutes to hours.

To get a clearer picture, the top 40 most common sequences are taken and used to create clusters of drivers. Data was normalized to ratios instead of absolute values. Following patterns are used as feature variables (R=request, V=verified): V..............., R..............., RV..............., R..V............, R.V............, R...V..........., R....V.........., R........V......, R...........V....., R.....V........., R.........V...., R.....V......., R..........V..., R.......V......., R.............V.., R...........V., FV.............R............V, F.V..........., R..RV.........., F..V.........., F..........., RRV..........., R.....RV........., RR............, R.RV............, RR.V............, R....RV........., R..............R, R.....RV......., F...V............, R..R............, R.............R., R.R.V..........., RR..V..........., RRRV............, R.R............, R..R.V.........., R...........R.., R...R...........

Each driver has an entry (feature value) for each of the above patterns, depending on whether he or she has participated in a selfie verification having that pattern. This means that a driver account can exhibit one or more of the above patterns, i.e. each driver has entries for multiple of these patterns (wherein entries may be zero, depending on whether they occurred for the driver). In some instances a driver may selfie verify himself or herself right away (pattern V........), other cases he or she may wait (short wait: RV.....). In other cases the same driver may wait a while (long wait R......V). Thus, each pair of data point (request->verification) included for the driver in the data contributes to one of the feature values of the driver. Suspicious drivers usually exhibit long wait for verification once on a while. They do not necessarily do it every time. Occasionally a driver using an account which is not his or hers (i.e. a fake driver) may get asked for a selfie when he or she is far away from the account owner. Thus, the fake driver will have to drive back to owner's home. A R........V pattern can be expected for such cases.

As another example, let the pattern be ‘R....RRF.......V’: the request was made initially for selfie, the driver kept ignoring it, eventually after three hours the fake driver tried a selfie verification and it failed. Then the fake driver had a long pause of many hours, eventually a successful verification happened, presumably the fake driver drove to the legit driver's place.

According to various embodiments, the drivers are clustered using these entries (feature values) for the various patterns. This means that there may be a table having a row for each driver, and 40 columns. Each of the 40 columns represent one of the most common sequences (i.e. the above patterns). For example, V......... . sequence is most common, where drivers selfie verify themselves within 15 seconds, then R....... . where selfie request is never answered within 8 hours, followed by RV where driver selfie verifies within 30 seconds, and so on. Rows are normalized to represent fraction of total cases for that driver in the data. In this fashion a table with 1,040,173 drivers, 40 columns of their selfie pattern is assembled.

Clustering shows that drivers who were involved in suspicious activities separate themselves out from regular drivers of good standing.

Clusters are for example generated by using k-means. For the exemplary data, k=8 was chosen since higher number of clusters only reduce the summed squared error (SSE) by a relatively small amount (it decreases rapidly until k=5 and then the slope gets increasingly smaller).

To understand clusters population composition the top three feature contributors for each cluster are considered:

Cluster #0, population: 30,162

R...............: 91.52027071538294

R..........V....: 0.6663949179337547

R........V.....: 0.6426418494427386

Cluster #1, pop: 738,189

V..............: 99.63681608438738

R...............: 0.06744260702821739

FV............: 0.035550546138602715

Cluster #2, pop: 141,524

V...............: 60.76066567556124

R..V............: 4.291151987458664

R.V..............: 3.871466998934899

Cluster #3, pop: 57,030

R.V............: 9.62941352186899

R.........V.....: 7.5803510027022565

R........V......: 7.17587344916754

Cluster #4, pop: 49,455

V...............: 51.14668522168341

R...............: 42.186882781357866

R...........V...: 0.49869819139111576

Cluster #5, pop: 5,726

R..V...........: 83.85025871279514

R...............: 2.7521999591819544

R.V............: 1.595195505240228

Cluster #6, pop: 13,666

RV............: 61.50764843690036

V...............: 31.199124627199804

R.V...........: 1.1461644442691485

Cluster #7, pop: 4,421

R...V..........: 84.50600039290718

R...............: 2.777712765542241

R.V............: 1.4471723578376734

Looking at the cluster features, it seems that the drivers of Clusters #1, #2, #4 and #6 are comporting themselves correctly. Cluster #0 (3%) contains drivers which behave in a weird manner by not react to selfie request. Drivers of Clusters #5, 7 seem to comport themselves correctly but have some overlap with suspicious activity. Drivers of Cluster #3 show a suspicious behaviour: their selfie patterns show high variation and include patterns where taking the selfie was delayed for a long time. It includes in fact a driver with major criminal behaviour included in the exemplary data set.

According to various embodiments, in accordance with the data analysis, drivers showing routine behaviour of delaying selfies are targeted for a repeat selfie loop with higher frequency, i.e. are more often requested to provide a selfie. This may for example be deployed in one region and a data analysis as above may be done for comparison.

Specifically, according to various embodiments, a driver is marked as suspicious if based on the time the driver takes to react to a selfie request, i.e. if the time exceeds a predetermined threshold (e.g. above 15 minutes).

So, an authentication server (e.g. part of server 203 or implemented by server 203 among other functions) performs detection of suspicious drivers (and flagging of detected drivers) and, depending on whether a driver is suspicious, triggering the request for a selfie.

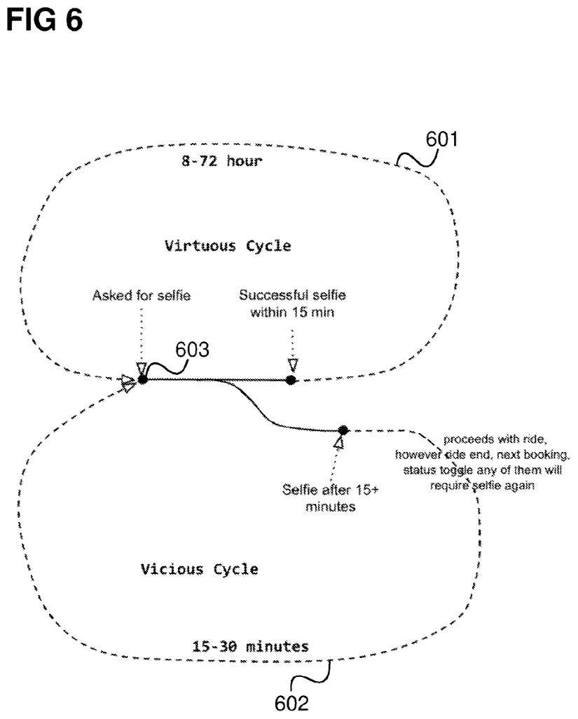

FIG. 6 illustrates a process to request selfies depending on a driver being suspicious due to having taken too long to react to an earlier selfie request.

The authentication server sends selfie requests 205 to a driver 201 according to a first loop 601 as long as the driver reacts to selfie requests 603 within 15 minutes successfully (i.e. with a selfie that matches the respective reference picture). According to the first loop 601, the authentication server requests the driver to provide a selfie according to a certain request interval, e.g. with 8-72 hours length.

If the driver 201 takes longer than 15 minutes to provide a selfie, the authentication server sends selfie requests 205 to the driver 201 according to a second loop 602. This means that for one or more selfie requests (e.g. until the driver has provided successful selfies for a certain number of requests) the authentication server sends the request upon events which usually occur much faster than the request interval of the first loop. For example, according to the second loop 602, the authentication server sends a selfie request to the driver
A. when the drivers toggles his or her availability state from not available to available.
B. when the driver has just completed a transport task. For this to work smoothly the authentication server may ensure that drivers flagged as suspicious do not have upcoming transport tasks assigned to them so as not to interfere with task allocation.

The authentication server may also apply, before requesting a selfie, a time buffer measured from the previous selfie request so as to maximize the probability that the selfie is requested while the driver is sufficiently away from the account owner, if it is a fake driver.

A driver may also be flagged suspicious (and be treated according to the second loop 602) according to other criteria such as
a number of passengers which report a different vehicle or a different driver for their rides than expected according to the driver's profile for the last few days exceeds a threshold.
the frequency with which the driver has switched phones for the last few days exceeds a threshold.

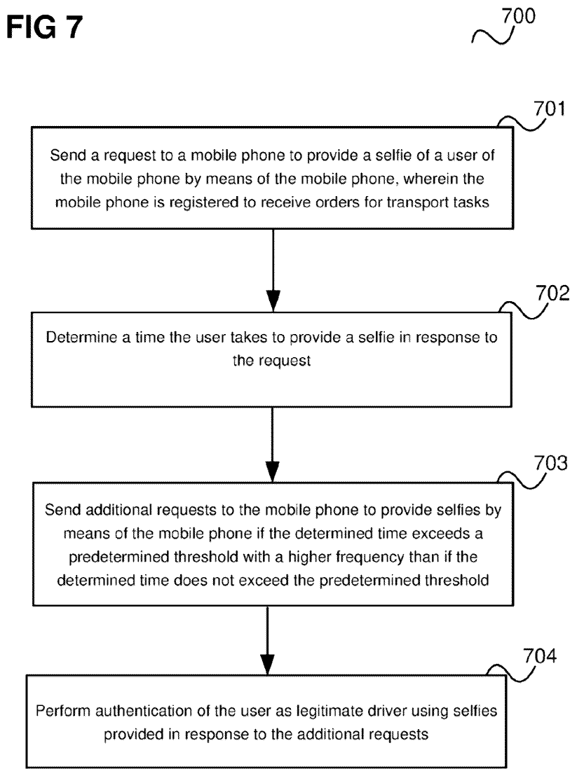

In summary, according to various embodiments, a method is provided as illustrated in FIG. 7.

FIG. 7 shows a flow diagram 700 illustrating a method for authenticating a driver of a vehicle.

In 701, a request is sent to a mobile phone to provide a selfie of a user of the mobile phone by means of the mobile phone, wherein the mobile phone is registered to receive orders for transport tasks.

In 702, a time the user takes to provide a selfie in response to the request is determined. This selfie may be used for a first driver authentication.

In 703, additional requests are sent to the mobile phone to provide selfies by means of the mobile phone if the determined time exceeds a predetermined threshold with a higher frequency than if the determined time does not exceed the predetermined threshold.

In 704, driver authentication is performed, i.e. authentication of the user as legitimate driver is performed using selfies provided in response to the additional requests.

According to various embodiments, in other words, selfies are requested more often from a user if the user takes too long to respond to a selfie request.

The approach of FIG. 7 allows reducing account sharing substantially (experiments show up to 75%). This is of high importance for an e-hailing service since, in particular, it improves how the service is perceived by its consumer base (i.e. improves trustworthiness), as drivers pretending to be someone else are typically also involved in criminal behaviour. On top of that, passengers may lose confidence if a driver's face does not match with the picture of a booked driver in an e-hailing app.

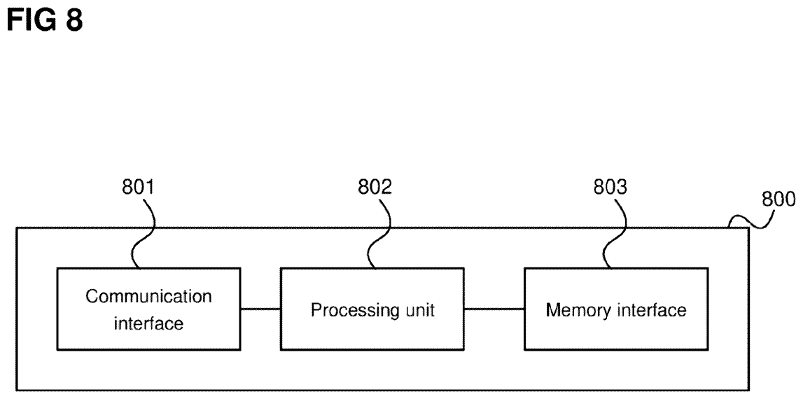

The method of FIG. 7 is for example carried out by an authentication server as illustrated in FIG. 8.

FIG. 8 shows an authentication server 800 according to an embodiment.

The authentication server 800, e.g. implemented by a server computer, includes a communication interface 801 (e.g. configured to receive requests for transport tasks). The authentication server 800 further includes a processing unit 802 and a memory 803. The memory 803 may be used by the processing unit 802 to store, for example, account data in particular reference pictures and logs of the times users took to respond to selfie requests. The authentication server 800 is configured to perform the method of FIG. 7. For example, the memory 803 stores program code which makes the authentication server 800 perform the method of FIG. 7.

The methods described herein may be performed and the various processing or computation units and the devices and computing entities described herein may be implemented by one or more circuits. In an embodiment, a “circuit” may be understood as any kind of a logic implementing entity, which may be hardware, software, firmware, or any combination thereof. Thus, in an embodiment, a “circuit” may be a hard-wired logic circuit or a programmable logic circuit such as a programmable processor, e.g. a microprocessor. A “circuit” may also be software being implemented or executed by a processor, e.g. any kind of computer program, e.g. a computer program using a virtual machine code. Any other kind of implementation of the respective functions which are described herein may also be understood as a “circuit” in accordance with an alternative embodiment.

While the disclosure has been particularly shown and described with reference to specific embodiments, it should be understood by those skilled in the art that various changes in form and detail may be made therein without departing from the spirit and scope of the invention as defined by the appended claims. The scope of the invention is thus indicated by the appended claims and all changes which come within the meaning and range of equivalency of the claims are therefore intended to be embraced.

1. A method for authenticating a driver of a vehicle, comprising:
sending a request to a mobile phone to provide a selfie of a user of the mobile phone by means of the mobile phone, wherein the mobile phone is registered to receive orders for transport tasks;
determining a time the user takes to provide a selfie in response to the request; sending additional requests to the mobile phone to provide selfies by means of the mobile phone if the determined time exceeds a predetermined threshold with a higher frequency than if the determined time does not exceed the predetermined threshold; and
performing authentication of the user as legitimate driver using selfies provided in response to the additional requests.

2. The method of claim 1, comprising, if the determined time exceeds the predetermined threshold, triggering the sending of an additional request at each of a set of predetermined events.

3. The method of claim 2, comprising avoiding triggering sending an additional request by the predetermined events if the determined time does not exceed the predetermined threshold.

4. The method of claim 3, wherein the predetermined events comprises the user toggling an availability state for taking transport tasks from not available to available and/or the user having completed a transport task.

5. The method of claim 1, wherein the predetermined threshold is in the range of 5 minutes to 30 minutes.

6. The method of claim 1, wherein the mobile phone is registered under a user account to receive orders for transport tasks and the method comprises flagging the user account as suspicious if the determined time exceeds the predetermined threshold and sending additional requests to the mobile phone if the account is flagged as suspicious than if the account is not flagged as suspicious.

7. The method of claim 6, comprising flagging the account as suspicious if a number of passengers which report a different vehicle or a different driver for their rides than expected according to the user's profile within a first predetermined period exceeds a first further predetermined threshold and/or comprising flagging the account as suspicious if the frequency with which different mobile phones have been used to register under the user account within a second predetermined period exceeds a second further predetermined threshold.

8. The method of claim 1, comprising sending the additional requests to the mobile phone with a minimum time buffer period between the sending of the additional requests.

9. The method of claim 8, wherein the time buffer period is in the range of 5 minutes to 30 minutes.

10. The method of claim 1, comprising sending an additional request after a predetermined request interval if the determined time does not exceed the predetermined threshold.

11. An authentication server configured to perform the method of claim 1.

12. A computer program element comprising program instructions, which, when executed by one or more processors, cause the one or more processors to perform the method of claim 1.

13. A computer-readable medium comprising program instructions, which, when executed by one or more processors, cause the one or more processors to perform the method of claim 1.
